(19) United States
(12) Patent Application Publication
(10) Pub. No.: US 2022/0315467 A1
(43) Pub. Date: Oct. 6, 2022

(54) BACILLUS METHYLOTROPHICUS STRAIN AND USE THEREOF FOR DEGRADING MICORPOLLUTANT IN ENVIRONMENT

(71) Applicant: NANJING UNIVERSITY, Jiangsu (CN)

(72) Inventors: Kailong HUANG, Jiangsu (CN); Xuxiang ZHANG, Jiangsu (CN); Lin YE, Jiangsu (CN); Hongqiang REN, Jiangsu (CN)

(73) Assignee: NANJING UNIVERSITY, Jiangsu (CN)

(21) Appl. No.: 17/617,937

(22) PCT Filed: Jan. 13, 2020
(86) PCT No.: PCT/CN2020/071679

§ 371 Date: Dec. 10, 2021

(30) Foreign Application Priority Data
Jan. 8, 2020 (CN) 202010017229.1

(51) Int. Cl. C02F 3/34; C02F 3/12; C12N 1/20

(52) U.S. Cl. CPC C02F 3/34; C02F 3/12; C12N 1/205; C02F 2101/34

(57) ABSTRACT

The present invention discloses a Bacillus methylotrophicus strain named Bacillus methylotrophicus BP1.1, which was deposited in China Center for Type Culture Collection under Deposit No. CCTCC M 20191078 on Dec. 20, 2019. The present invention further discloses the use of the Bacillus methylotrophicus strain for degrading benzophenone ultraviolet sunscreens. By domesticating the activated sludge of the domestic sewage treatment plant step-by-step, the present invention provides a Bacillus methylotrophicus BP1.1 strain which has high efficiency in removing benzophenone ultraviolet sunscreens in water environment.

### TECHNICAL FIELD

The present invention relates to the field of wastewater treatment, and particularly, to a Bacillus methylotrophicus strain and use thereof for degrading benzophenone ultraviolet sunscreens in water.

### BACKGROUND

Benzophenones (BPs) ultraviolet sunscreens are widely used in personal care products due to their good safety, sunscreen effect, and cost-efficiency. Most ultraviolet sunscreens have good durability. With the increase of daily usage amount, benzophenone ultraviolet sunscreens continuously enter environmental water, but cannot be completely removed in urban wastewater treatment plants, and will eventually converge into rivers and lakes and continuously accumulate. Thus the detection of ultraviolet sunscreens in water environments is also becoming more common. Among them, benzophenone ultraviolet sunscreens are lipophilic substances, have endocrine disrupting effect and bioaccumulation, and are widely exist in the environment, becoming an emerging micropollutant.

Photochemical conversion is an important way for converting ultraviolet sunscreens in natural water body. However, the reaction rate and the pathway for photochemical conversion are influenced by pH of water environment, soluble substances, etc., and BPs can produce more eco-toxic metabolites under illumination. Advanced oxidation technology is to degrade benzophenone by using a strong oxidizing substance with hydroxyl radical HO generated in the reaction process. The most commonly reported technologies at present include UV/H₂O₂, O₃/H₂O₂, UV/O₃, Fenton and the like, etc., the chemical reagents added in the advanced oxidation reaction and the partial products generated in the conversion process have potential environmental risks. Microbial degradation is the only way for eventually mineralizing the organic matters in the nature and plays a key role in treating environmental organic pollutants. Since the biodegradability of BPs is relatively lower, fewer studies have focused on the microbial degradation of BPs. The latest studies discovered that the biodegradation rate of BPs in activated sludge in the aerobic condition may reach 60%, but the degradation requires a longer period, generally 10-20 days.

### SUMMARY OF INVENTION

The present invention is intended to solve the technical problem of providing a microorganism, i.e., Bacillus methylotrophicus, which can rapidly degrade benzophenone ultraviolet sunscreens in water environment under aerobic conditions.

Technical Scheme: A Bacillus methylotrophicus strain named as Bacillus methylotrophicus BP1.1, with the classification name of Bacillus methylotrophicus and the strain number of BP1.1 was deposited in China Center for Type Culture Collection (Deposit No. CCTCC M 20191078) on Dec. 20, 2019.

The Bacillus methylotrophicus BP1.1 strain grows well in LB culture medium under aerobic conditions at 30° C. The colonies have a round shape, a diameter of 0.2-1 mm, a light pink color, an opaque appearance and a dry and smooth surface. The stain is gram-negative, and demonstrates a short-rod shape under the microscope.

The present invention is further intended to solve the technical problem of providing the application of the Bacillus methylotrophicus strain for degrading a micropollutant in the environment.

The micropollutant is a benzophenone ultraviolet sunscreen; the benzophenone ultraviolet sunscreen is 2,4-dihydroxybenzophenone (BP-1).

The application is as follows: the Bacillus methylotrophicus BP1.1 strain is inoculated into an expansion culture medium at an inoculation amount of 1%, and incubated under aerobic conditions at 28-30° C. for 24-48 h to obtain a BP1.1 expansion product; the expanded culture product is inoculated into the wastewater containing benzophenone ultraviolet sunscreen for treatment. The inoculation amount of Bacillus methylotrophicus BP1.1 strain in the wastewater is 1%ₒ-2%ₒ, and the mass concentration of the benzophenone ultraviolet sunscreen in wastewater is 10 mg/L.

For using the strain, the temperature of the wastewater containing benzophenone ultraviolet sunscreen is 20-30° C., preferably 28° C., and the pH is 7.3-8.0, preferably 7.4, under these conditions, the degradation rate of the benzophenones in the wastewater by Bacillus methylotrophicus BP1.1 can be facilitated.

Beneficial Effects: the biological degradation ratio of benzophenone ultraviolet sunscreens by the Bacillus methylotrophicus strain under aerobic conditions may reach 100%, and significantly shortened the time for degradation; the present invention can rapidly and efficiently degrade the emerging micropollutant BP-1 in wastewater through a biological method, which has great significance for treating the emerging micropollutant in wastewater.

### Solution to Problem

### BRIEF DESCRIPTION OF DRAWINGS

FIG. 1 illustrated a microscopic morphology of gram-stained Bacillus methylotrophicus BP1.1 in Example 2 of the present invention;

FIG. 2 illustrated the phylogenetic tree of Bacillus methylotrophicus BP1.1 in Example 2 of the present invention;

FIG. 3 illustrated the degradation rate of 2,4-dihydroxybenzophenone by Bacillus methylotrophicus BP1.1 in Example 3 of the present invention.

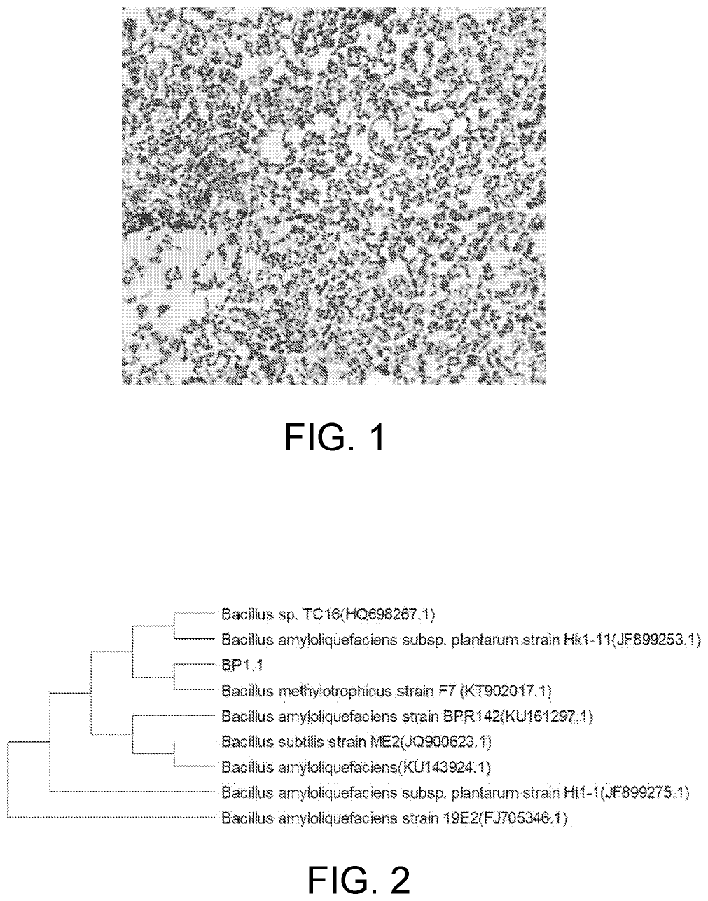

### DESCRIPTION OF EMBODIMENTS

The technical solutions of the present invention are further described in detail below in conjunction with drawings and specific embodiments.

### Example 1

In the present invention, Bacillus methylotrophicus BP1.1 was obtained from activated sludge at aerobic stage of a domestic sewage treatment plant in Nanjing.

The specific procedures are as follows:

(1) Activated sludge was obtained at aerobic stage from a domestic sewage treatment plant in Nanjing;

(2) 250 mL of the sludge-water mixture was added into a conical flask, stirred with a magnetic stirrer at room temperature, and added with 5 mg of 2,4-dihydroxybenzophenone every day; the supernatant was removed before each addition and was detected for the target pollutant 2,4-dihydroxybenzophenone, and the mixture was filled up to the volume with tap water; the strain was continuously acclimated until the target pollutant was not detected in the solution;

(3) the sludge obtained in step (2) was gradient diluted and spread on an inorganic salt culture medium containing 2,4-dihydroxybenzophenone, and incubated for 3-5 days at 30° C. to get individual colonies; the mass concentration of 2,4-dihydroxybenzophenone in the inorganic salt culture medium was 50 mg/L;

(4) individual colonies in step (3) was selected, isolated by streaking on an inclined plane, and preserved to obtain an isolated strain.

The culture medium for expanding the isolated strain was one of LB culture medium, inorganic salt culture medium or beef peptone culture medium.

The LB culture medium comprised the following components by mass per liter: 5.0 g of yeast powder, 10 g of sodium chloride and 10 g of peptone, pH 7.0-7.5.

The inorganic salt culture medium comprised the following components by mass per liter: 10-50 mg of 2,4-dihydroxybenzophenone, 1.6 g of sodium hydroxide and 6.8 g of monopotassium phosphate, pH 7.3-7.5. The Bacillus methylotrophicus BP1.1 strain can grow and propagate using 2,4-dihydroxybenzophenone as the only carbon source.

The beef peptone culture medium comprised the following components by mass per liter: 3 g of beef, 10 g of peptone and 5 g of sodium chloride, pH 7.2-7.6.

The Bacillus methylotrophicus BP1.1 strain obtained in step (4) was inoculated into an expansion culture medium with an inoculation amount of 1% (volume fraction) for expansion, and was incubated under aerobic conditions at 28-30° C. for 24-48 h to get a BP1.1 expansion culture product.

### Example 2

The strain selected in Example 1 was identified.

In Example 1 of the present invention, a BP1.1 strain with the best degradation effect and the fastest growth rate was obtained by screening. The BP1.1 was identified as Bacillus methylotrophicus.

The Bacillus methylotrophicus BP1.1 strain grew well in LB culture medium under aerobic conditions at 30° C. The colony had a round shape, a diameter of 0.2-1 mm, a light pink color, an opaque appearance and a dry and smooth surface. The stain was gram-negative, and demonstrated a short-rod shape under a microscope.

The complete sequence of the 16S rRNA of the BP1.1 strain obtained by PCR amplification and Sanger sequencing is as follows (SEQ ID NO. 1):

agggggcggggcggcgtgctatacatgcaagtcgagcggacagatggga
gcttgctccctgatgttagcggcggacgggtgagtaacacgtgggtaac
ctgcctgtaagactgggataactccgggaaaccggggctaataccggat
ggttgtctgaaccgcatggttcagacataaaaggtggcttcggctacca
cttacagatggacccgcggcgcattagctagttggtgaggtaacggctc
accaaggcgacgatgcgtagccgacctgagagggtgatcggccacactg
ggactgagacacggcccagactcctacgggaggcagcagtagggaatct
tccgcaatggacgaaagtctgacggagcaacgccgcgtgagtgatgaag
gttttcggatcgtaaagctctgttgttagggaagaacaagtgccgttca
aatagggcggcaccttgacggtacctaaccagaaagccacggctaacta
cgtgccagcagccgcggtaatacgtaggtggcaagcgttgtccggaatt
attgggcgtaaagggctcgcaggcggtttcttaagtctgatgtgaaagc
ccccggctcaaccggggagggtcattggaaactggggaacttgagtgca
gaagaggagagtggaattccacgtgtagcggtgaaatgcgtagagatgt
ggaggaacaccagtggcgaaggcgactctaggtagtaactgacgctgag
gagcgaaagcgtggggagcgaacaggattagataccaggtagtccacgc
cgtaaacgatgagtgctaagtgttagggggtttccgccccttagtgagc
agctaacgcattaagcactccgcctggggagtacggtcgcaagactgaa
actcaaaggaattgacgggggcccgcacaagcggtggagcatgtggttt
aattcgaagcaacgcgaagaaccttaccaggtcttgacatcctctgaca
atcctagagataggacgtccccttcgggggcagagtgacaggtggtgca
tggttgtcgtcagctcgtgtcgtgagatgttgggttaagtcccgcaacg
agcgcaacccttgatcttagttgccagcattcagttgggcactctaagg
tgactgccggtgacaaaccggaggaaggtggggatgacgtcaaatcatc
atgccccttatgacctgggctacacacgtgctacaatggacagaacaaa
gggcagcgaaaccgcgaggttaagccaatcccacaaatagttctcagtt
cggatcgcagtctgcaactcgactgcgtgaagctggaatcgctagtaat
cgcggatcagcatgccgcggtgaatacgttcccgggccttgtacacacc
gcccgtcacaccacgagagtttgtaacacccgaagtcggtgaggtaacc
tttatggagccagccgccgaaggtgaacccggagttg

The sequencing result of BP1.1 was submitted to GenBank for BLAST alignment, and strain sequences with more than 98% similarity to base sequences of BP1.1 were selected from the BLAST results. Clustal W multiple sequence alignment was performed, and a phylogenetic tree was established using MAGE 7.0, indicating that the genetic relationship between BP1.1 and Bacillus methylotrophicus was the nearest. Thus the strain BP1.1 was identified as Bacillus methylotrophicus.

### Example 3

The degradation rate of the pollutant 2,4-dihydroxybenzophenone by the Bacillus methylotrophicus BP1.1 strain was measured. The method comprised the following steps:

(1) the expansion culture of BP1.1 was centrifuged at 6000 rpm for 10 min, and the supernatant was removed to get the strain;

(2) the strain obtained in step (1) was resuspended and inoculated into a water solution containing 2,4-dihydroxybenzophenone with an inoculation amount of 1.8%ₒ; the mass concentration of 2,4-dihydroxybenzophenone in the solution was 10 mg/L; a group without adding BP1.1 was set as a control;

(3) the control group and the treatment group in step (2) were shaken on a shaker under aerobic conditions at 150 rpm and 28° C.;

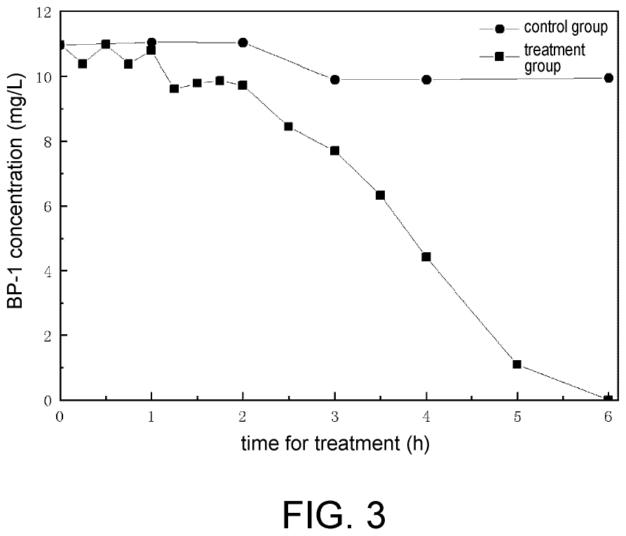

(4) the concentration of remaining pollutant in the treatment and control group of step (3) were regularly sampled and measured. The results were shown in FIG. 3.

As shown in FIG. 3, the BP1.1 strain can efficiently degrade 2,4-dihydroxybenzophenone in water. 2,4-Dihydroxybenzophenone at 10 mg/L could be completely degraded within 6 h. In the first 2 h of treatment, the BP1.1 strain had a lower degradation rate due to the adaptability period in the degradation system; after the adaptation (2-5 h), the degradation process came into a rapid stage; in the late degradation stage (5-6 h), the degradation rate gradually became slower due to the decrease of substrate concentration.

By domesticating the activated sludge at aerobic stage of a domestic sewage treatment plant step-by-step, the present invention provides a Bacillus methylotrophicus BP1.1 strain with high removal capacity for benzophenone ultraviolet sunscreens. The biological degradation rate of benzophenone ultraviolet sunscreens by the Bacillus methylotrophicus strain under aerobic conditions may reach 100%, and it can significantly shorten the time for degradation.

1. A Bacillus methylotrophicus strain, wherein the strain is named as Bacillus methylotrophicus BP1.1, and deposited in China Center for Type Culture Collection under Deposit No. CCTCC M 20191078 on Dec. 20, 2019.

2-9. (canceled)

10. A method for degrading a micropollutant in an environment, comprising:
using a Bacillus methylotrophicus strain, wherein the strain is named as Bacillus methylotrophicus BP1.1, and deposited in China Center for Type Culture Collection under Deposit No. CCTCC M 20191078 on Dec. 20, 2019.

11. The method according to claim 10, wherein the micropollutant is a benzophenone ultraviolet sunscreen, and the benzophenone ultraviolet sunscreen is 2,4-dihydroxybenzophenone.

12. The method according to claim 11, wherein the Bacillus methylotrophicus strain degrades the benzophenone ultraviolet sunscreen in an aerobic condition.

13. The method according to claim 10, wherein the Bacillus methylotrophicus BP1.1 strain is inoculated into an expansion culture medium, and incubated in an aerobic condition at 28-30° C. for 24-48 h to obtain a BP1.1 expansion product, and the expanded culture product is inoculated into a wastewater containing a benzophenone ultraviolet sunscreen for treatment.

14. The method according to claim 13, wherein the inoculation amount of Bacillus methylotrophicus BP1.1 in the wastewater is 1%ₒ-2%ₒ.

15. The method according to claim 13, wherein the mass concentration of the benzophenone ultraviolet sunscreen in the wastewater is 10 mg/L.

16. The method according to claim 13, wherein the temperature of the wastewater is 20-30° C., and the pH of the wastewater is 7.3-8.0.

17. The method according to claim 13, wherein the expansion culture medium is one of a LB culture medium, an inorganic salt culture medium or a beef peptone culture medium.


SEQUENCE LISTING (1 sequence)

SEQ ID NO: 1 - DNA, 1463 bp, Bacillus methylotrophicus
agggggcggggcggcgtgctatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgaacccggagttg